SEQUENCE LISTING (9 sequences)

SEQ ID NO: 1 - DNA, 8 bp, Artificial Sequence
  SV40 large T antigen nuclear localization signal
akkkrkvg

SEQ ID NO: 2 - DNA, 35 bp, Artificial Sequence
  ratIR1
cctggtacagcctggaataatataactgtttatac

SEQ ID NO: 3 - DNA, 34 bp, Homo Sapien
cctggtacagcctgaaataatgtattgtttatcc

SEQ ID NO: 4 - DNA, 23 bp, Artificial Sequence
  rSHP
gatccctgggttaataaccctgt

SEQ ID NO: 5 - DNA, 23 bp, Artificial Sequence
  mSHP
gatccctgggttaatgaccctgt

SEQ ID NO: 6 - DNA, 23 bp, Artificial Sequence
  hSHP
gatccctgagttaatgaccttgt

SEQ ID NO: 7 - DNA, 23 bp, Artificial Sequence
  mI-BABP
gatcttaaggtgaataaccttgg

SEQ ID NO: 8 - DNA, 22 bp, Artificial Sequence
  hI-BABP
gatcccaggtgaataacctcgg

SEQ ID NO: 9 - DNA, 23 bp, Artificial Sequence
  mSHPmut
gatccctggaataatgttcctgt

(19) United States
(12) Patent Application Publication
(10) Pub. No.: US 2004/0038862 A1
(43) Pub. Date: Feb. 26, 2004

(54) Identification of new therapeutic targets for modulating bile acid synthesis

(76) Inventors: Bryan James Goodwin, Durham, NC (US); Stacey Ann Jones, Durham, NC (US); Steven Anthony Kliewer, Durham, NC (US); Patrick Reed Maloney, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/343,289

(22) PCT Filed: Jul. 30, 2001
(86) PCT No.: PCT/US01/24203

(51) Int. Cl. G01N 33/53; G01N 33/567; A61K 38/17

(52) U.S. Cl. 514/2; 435/7.2

(57) ABSTRACT

Abstract: Methods for identifying compounds that modulate bile acid synthesis by assessing their ability to act as ligands for short heterodimerizing partner-1 or liver receptor homologue-1 are provided. Also provided are compositions containing these ligands as well as methods for administering these compositions to modulate bile acid synthesis and cholesterol and lipid homeostasis.

### FIELD OF THE INVENTION

A regulatory cascade of three orphan nuclear receptors, farnesoid X receptor (FXR), short heterodimerizing partner-1 (SHP-1), and liver receptor homologue-1 (LRH-1) has now been identified which provides a molecular basis for the coordinate repression of bile acid synthesis and cholesterol and lipid homeostasis. Specifically, it has been found that FXR induces expression of SHP-1 which represses expression of cytochrome P450 7A (CYP7A) by binding to LHR-1. CYP7A catalyzes the rate limiting step in bile acid biosynthesis. The present invention relates to the identification of these receptors as therapeutic targets and the development of ligands targeted to these receptors for use in modulating bile acid synthesis. In particular, the present invention relates to the identification of ligands which modulate the interaction of SHP-1 and LRH-1. Methods for using these ligands to modulate bile acid synthesis and cholesterol and lipid homeostasis are also provided.

### BACKGROUND OF THE INVENTION

Cholesterol is essential for a number of cellular processes, including membrane biogenesis and steroid hormone and bile acid biosynthesis. It is the building block for each of the major classes of lipoproteins found in cells of the human body. Accordingly, cholesterol biosynthesis and catabolism are highly regulated and coordinated processes. A number of diseases and/or disorders have been linked to alterations in cholesterol metabolism or catabolism including atherosclerosis, gall stone formation, and ischemic heart disease. An understanding of the pathways involved in cholesterol homeostasis is essential to the development of useful therapeutics for treatment of these diseases and disorders represents a major pathway for cholesterol elimination from the body, accounting for approximately half of the daily excretion. These cholesterol metabolites are formed in the liver and secreted into the duodenum of the intestine, where they have important roles in the solubilization and absorption of dietary lipids and vitamins. Most bile acids (approximately 95%) are subsequently reabsorbed in the ileum and returned to the liver via the enterohepatic circulatory system.

Cytochrome P450 7A (CYP7A) is a liver specific enzyme that catalyzes the first and rate-limiting step in one of the two pathways for bile acid biosynthesis (Chiang, J. Y. L. 1998. Front. Biosci. 3:176-193; Russell, D. W. and K. D. Setchell. 1992. Biochemistry 31:4737-4749). The gene encoding CYP7A is regulated by a variety of endogenous, small, lipophilic molecules including steroid and thyroid hormones, cholesterol, and bile acids. Notably, CYP7A expression is stimulated by cholesterol feeding and repressed by bile acids. Thus, CYP7A expression is both positively (stimulated or induced) and negatively (inhibited or repressed) regulated.

CYP7A expression is regulated by several members of the nuclear receptor family of ligand-activated transcription factors (Chiang, J. Y. L. 1998. Front. Biosci. 3:176-193; Gustafsson, J. A. 1999. Science 284:1285-1286; Russell, D. W. 1999. Cell 97:539-542). Recently, two nuclear receptors, the liver X receptor (LXR ; NR1H3; Apfel, R. et al. 1994. Mol. Cell. Biol. 14:7025-7035; Willy, P. J. et al. 1995. Genes Devel. 9:1033-1045) and the farnesoid X receptor (FXR; NR1H4; Forman, B. M. et al. 1995. Cell 81:687-693; Seol, W. et al. 1995. Mol. Endocrinol. 9:72-85) were implicated in the positive and negative regulation of CYP7A (Peet, D. J. et al. 1998. Curr. Opin. Genet. Develop. 8:571-575; Russell, D. W. 1999. Cell 97:539-542). Both LXR and FXR are abundantly expressed in the liver and bind to their cognate hormone response elements (Mangelsdorf, D. J. and R. M. Evans. 1995. Cell 83:841-850). LXR is activated by the cholesterol derivative 24,25(S)-epoxycholesterol and binds to a response element in the CYP7A promoter (Lehmann, J. M. et al. 1997. J. Biol. Chem. 272:3137-3140). CYP7A is not induced in response to cholesterol feeding in mice lacking LXR (Peet, D. J. et al. 1998. Cell 93:693-704). Moreover, these animals accumulate massive amounts of cholesterol in their livers when fed a high cholesterol diet. These studies establish LXR as a cholesterol sensor responsible for positive regulation of CYP7A expression.

Bile acids stimulate the expression of genes involved in bile acid transport such as the intestinal bile acid binding protein (I-BABP) and repress CYP7A as well as other genes involved in bile acid biosynthesis such as CYP8B (which converts chenodeoxycholic acid to cholic acid), and CYP27 (which catalyzes the first step in the alternative “acidic” pathway for bile acid synthesis) (Javitt, N. B. 1994. FASEB J. 8:1308-1311; Russell, D. W. and K. D. Setchell. 1992. Biochemistry 31:4737-4749). Recently, FXR was shown to be a bile acid receptor (Makishima, M. et al. 1999. Science 284:1362-1365; Parks, D. J. et al. 1999. Science 284:1365-1368; Wang, H. 1999. Mol. Cell 3:543-553). Several different bile acids, including chenodeoxycholic acid and its glycine and taurine conjugates were demonstrated to bind to and activate FXR at physiologic concentrations. In addition, DNA response elements for the FXR/RXR heterodimer were identified in both the human and mouse I-BABP promoters, indicating that FXR mediates positive effects of bile acids on I-BABP expression (Grober, J. et al. 1999. J. Biol. Chem. 274:29749-29754; Makishima, M. et al. 1999. Science 284:1362-1365). Further, the rank order of bile acids that activate FXR correlates with that for repression of CYP7A in a hepatocyte-derived cell line (Makishima, M. et al. 1999. Science 284:1362-1365). Thus, these studies indicate that FXR also has a role in thee negative effects of bile acids on gene expression.

However, the molecular mechanism of bile acid-mediated repression of CYP7A, and specifically the role of FXR has been unclear. Since the CYP7A promoter lacks a strong FXR/RXR binding site (Chiang, J. Y. and D. Stroup. 1994. J. Biol. Chem269:17502-17507; Chiang, J. Y. et al. 2000. J. Biol. Chem. 275:10918-10924), it is unlikely that the effect is from the direct interaction of FXR.

A ligand which selectively binds and activates FXR has been identified. Using this ligand it has been demonstrated that the human orphan nuclear receptor, FXR, interacts with a nuclear receptor, short heterodimerizing partner-1 (SHP-1). Further, it has now been demonstrated that SHP-1 interacts with LRH-1 to modulate expression of CYP7A. Accordingly, these three receptors are part of a regulatory cascade for coordinate repression of bile acid synthesis and cholesterol and lipid homeostasis.

### SUMMARY OF THE INVENTION

An object of the present invention is to provide methods for identifying new therapeutic agents which modulate bile acid synthesis. These agents comprise ligands which interact with short heterodimerizing partner-1 (SHP-1) or liver receptor homologue-1 (LRH-1) to modulate expression of genes involved in bile acid synthesis. In a preferred embodiment of the present invention, the agents comprise ligands which modulate the interaction of SHP-1 with LRH-1. Another object of the present invention is to provide a method for modulating bile acid synthesis in a patient in need thereof which comprises administering to the patient a composition comprising a ligand for short heterodimerizing partner-1 (SHP-1) or liver receptor homologue-1 (LRH-1). In a preferred embodiment, the composition comprises a ligand which modulates the interaction of SHP-1 with LRH-1.

This technology can thus be used to affect bile acid and cholesterol and lipid homeostasis such that ultimately cholesterol and lipid levels are modified and to treat diseases in which regulation of bile acid, cholesterol and lipid levels is important.

### DETAILED DESCRIPTION OF THE INVENTION

Bile acids are cholesterol metabolites formed in the liver and secreted into the duodenum of the intestine wherein assist in the solubilization and absorption of dietary lipids and vitamins. Thus, bile acids have an important role not only in regulating cholesterol homeostasis, but also in regulating lipid homeostasis. Modulators of bile acid synthesis can therefore be used in a variety of treatments including, but not limited to, inhibition of fatty acid absorption in the intestine for the treatment of dyslipidemia, obesity and associated diseases including atherosclerosis, inhibition of protein and carbohydrate digestion in the intestine for the treatment of obesity, and inhibition of de novo cholesterol biosynthesis in the liver for the treatment of disease related to elevated cholesterol levels including atherosclerosis and gall stones.

Bile acids repress the expression of genes involved in their biosynthesis, including cytochrome P450 7A (CYP7A) which catalyzes the rate limiting step in bile acid biosynthesis. A bile-acid regulatory cascade providing a molecular basis for the coordinate suppression of CYP7A and other genes involved in bile acid synthesis has now been identified. Using a potent, non-steroidal farnesoid X receptor (FXR) ligand, it has been demonstrated that FXR induces expression of short heterodimerizing protein 1 (SHP-1; NRB02), an atypical member of the nuclear receptor family that lacks a DNA binding domain. Further, it has now been demonstrated that SHP-1 represses expression of CYP7A by binding to the nuclear receptor liver receptor homologue 1 (LRH-1; NR5A2), which binds to a response element in the CY7A gene promoter. The interaction of SHP-1 and LRH-1 can also result in alterations of expression of other genes that these receptors aid in regulating, including genes involved in lipid absorption and digestion in the small intestine and lipid homeostasis in the liver. Examples of such genes include, but are not limited to, genes involved in bile acid transport, lipid absorption, cholesterol biosynthesis, proteolysis, amino acid metabolism, glucose biosynthesis, protein translation, electron transport and hepatic fatty acid metabolism. Thus, the identification of the SHP-1 and LRH-1 receptors being involved in this regulatory cascade serves as a basis for identifying and designing compositions useful in the modulation of bile acid synthesis and cholesterol and lipid homeostasis.

Accordingly, the present invention relates to the identification of ligands specific for SHP-1 or LHR-1 and methods of using these ligands in compositions for the modulation of bile acid synthesis as well as cholesterol homeostasis and lipid homeostasis. In a preferred embodiment of the present invention, the ligands modulate the interaction of SHP-1 with LRH-1. For purposes of the present invention, by “modulation”, “modulate”, or “modulator” it is meant to regulate, adjust or alter physiological conditions or parameters associated with SHP-1 and LRH-1. Thus, examples of modulation include, but are not limited to, the ligand either increasing or decreasing gene expression or activity of the SHP-1 or LRH-1 receptors identified in this biosynthetic cascade for bile acid synthesis, alterations in timing of expression of one or both of these receptors, increases or decrease in bile acid synthesis, and alterations in cholesterol and lipid homeostasis. By the term “ligand” it is meant a compound with, the pharmacologic activity to bind to and modulate a receptor in this biosynthetic cascade for bile acid synthesis. In a preferred embodiment, binding of the ligand to either the SHP-1 or LRH-1 receptor modulates the Ligands for use in the compositions of the present invention can be identified routinely through screening of libraries of compounds using assays such as the FRET assay as described in Parks, D. J. 1999. Science 284:1365-1368 and in WO 00/25134. This assay was used to identify a potent ligand for the FXR receptor. This ligand, referred to herein as GW4064, is depicted in Formula (I):

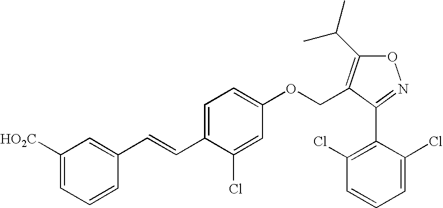

In contrast to bile acids such as chenodeoxycholic acid which bind to FXR with low (micromolar) affinities and interact with other proteins, the potent, selective FXR ligand, GW4064 binds to FXR with an EC₅₀ value of 15 nm. GW4064 also activates rodent and human FXR with EC₅₀ values of 80 and 90 nm, respectively, in CV-1 cells transfected with FXR expression vectors and a reporter driven by two copies of the hsp70 ecdysone receptor response element. Accordingly, this isoxazole of Formula I is 100-fold more I potent than chenodeoxycholic acid as an FXR agonist. GW4064 is also highly selective for FXR, activating only the FXR-GAL4 chimera in a panel of nuclear receptor binding assays wherein CV-1 cells were transfected with expression vectors for various GAL4-nuclear receptor ligand binding domain chimeras and the reporter plasmid (UAS)₅-tk-CAT.

Several recent studies have implicated FXR in the repression of CYP7A (Makishima et al. 1999 Science. 284:1362-5; Parks et al. 1999 Science 284:1365-8, Wang et al. 1999 Molecular Cell 3:543-53). Repression of expression of CYP7A by compounds such as bile acids is known to be part of a regulatory feedback loop that controls the rate of their biosynthesis from cholesterol (Russell, D. W. 1999. Cell 97:539-42; Russell, D. W. and K. D. Setchell, 1992. Biochemistry 31:4737-49). Accordingly, the effects of GW4064 on CYP7A expression were examined.

Treatment of animals with GW4064 was demonstrated to decrease CYP7A levels. Rats treated with GW4064 for 7 days showed a decrease in CYP7A expression levels as compared to vehicle treated rats. This decrease was still measurable despite the fact that the animals had been maintained on a normal light cycle and sacrificed during the daytime when CYP7A levels are known to be quite low. The ability of GW4064 to decrease CYP7A expression in a dose dependent fashion was confirmed in human hepatocytes.

As will be understood by those of skill in the art upon reading this disclosure, additional ligands which are selective for FXR and useful in compositions of the present invention can also be identified in accordance with the procedures described herein. Further, the structure of GW4064 provides a template for the design of new compounds with similar structures also expected to be selective ligands for FXR. Using this structure as a template both agonists and antagonists for FXR can be designed. The selectivity of these new compounds for FXR can be determined routinely by those of skill in the art based upon these teachings provided herein. Like GW4064, newly identified selective FXR ligands can also be used in the modulation of bile acid biosynthesis.

Using GW4064, SHP-1 has also been identified to be involved in the regulation FXR in the liver. RNA prepared from the livers of rats treated with GW4064 for 7 days exhibited a six-fold increase in SHP-1 expression as compared to RNA from vehicle-treated rats. GW4064 treatment also markedly increased SHP-1 expression in a dose-dependent manner in hepatocytes from both humans and rats. Results from these studies were similar to results from human hepatocytes treated with chenodeoxycholic acid, an endogenous FXR ligand; however, the endogenous ligand was much less potent than GW4064. The reciprocal relationship between regulation of SHP-1 and CYP7A expression, i.e., GW4064 and chenodeoxycholic acid repressed CYP7A expression at the same concentrations that were required for induction of SHP-1 expression, is indicative of FXR-mediated induction of SHP-1 being involved in repression of CYP7A expression. Further, scanning of the mouse, rat and human SHP-1 has revealed the presence of an FXR/RXR binding site within the SHP-1 promoter, which is indicative of the SHP-1 gene being directly regulated by FXR. Direct regulation of SHP-1 by FXR was confirmed in experiments in HepG2 cells transfected with an FXR expression plasmid and reporter plasmids under the control of either the rat or human SHP-1 promoter. Treatment of cells transfected with the FXR expression plasmid and either promoter with GW4064 resulted in a marked induction of reporter activity. In contrast, cells with no FXR or mutations in the SHP-1 promoter for the FXR/RXR binding site showed little to no induction.

Using a mammalian two-hybrid approach, experiments were then performed to determine the ability of SHP-1 to interact with a variety of nuclear receptors implicated in the regulation of CYP7A. CV-1 cells were transfected with an expression plasmid for a GAL4-SHP-1 chimera, the (UAS)₅-tk-CAT reporter and expression plasmids for chimeras between the strong transcriptional activation domain of VP16 and the isolated ligand binding domains of TR, RXR, RAR, LXR, COUP-TF, HNF4, and LRH-1. The GAL4-SHP-1 chimera had no activity on its own. Increased reporter activity was detected when GAL4-SHP-1 was co-expressed with RXR in the presence of its ligand 9-cis retinoic acid, demonstrating that this nuclear receptor interacts with SHP-1 in cells in a ligand-dependent fashion. Strong reporter activity was also detected when GAL4-SHP-1 was cotransfected with VP16-LRH-1, activity that was dependent on the presence of GAL4-SHP-1. Accordingly, these data demonstrate that SHP-1 interacts with LRH-1 in cells.

SHP-1 was also demonstrated to play a role in the repression of CYP7A expression. Cotransfection experiments were performed with a rat CYP7A luciferase reporter plasmid containing nucleotides −1573 to +36 of the rat CYP7A promoter, including a conserved LRH-1 binding site. Reporter activity was detected when CYP7A-LUC was introduced into HepG2 cells, demonstrating that the CYP7A promoter has basal activity. Cotransfection of increasing amounts of a LRH-1 expression plasmid resulted in a dose-dependent increase in reporter activity. The LRH-dependent reporter activity was completely blocked by the cotransfection of SHP-1 expression plasmid. Thus, these data demonstrate that SHP-1 can repress LRH-1-dependent activation of the CYP7A promoter.

Accordingly, compositions comprising ligands for SHP-1 can be used in the modulation of bile acid synthesis and cholesterol and lipid homeostasis. Further, as demonstrated herein, activation of the CYP7A promoter is also dependent on LRH-1. Thus, compositions comprising ligands selective to LRH-1 can also be used to modulate bile acid biosynthesis and cholesterol and lipid homeostasis. In a preferred embodiment of the present invention, the composition comprises a ligand which modulates the interaction of SHP-1 with LRH-1.

Screening of ligands that modulate the SHP-1/LRH-1 interaction can be performed using the mammalian two-hybrid approach described in the preceding paragraph. This approach identifies both SHP-1 modulators and LRH-1 modulators. Alternatively, a FRET-based interaction assay using the LRH-1 ligand binding domain and an interacting peptide from SHP-1 can be employed to identify ligands that modulate the LRH-1/SHP-1 interaction.

Compositions of the present invention comprising a ligand for SHP-1 or LHR-1 can be administered to a patient to modulate CYP7A expression levels, thereby modulating bile acid synthesis and cholesterol homeostasis. Ligands which activate FXR transcriptional activity, promote or strengthen the SHP-1/LRH-1 interaction, or inhibit LRH-1 transcriptional activity decrease expression levels of CYP7A, thereby modulating the rate of bile acid synthesis. Accordingly, the compositions of the present invention are useful in modulating cholesterol homeostasis as well as lipid homeostasis and in the treatment of diseases and disorders including, but not limited to, atherosclerosis, gall stones, ischemic heart disease, obesity, and dyslipidemia.

Dosing regimes, as well as selection of appropriate routes of administration for the compositions of the present invention can be determined routinely by one of skill in the art based upon in vitro and in vivo data generated in accordance with procedures such as described herein. It is preferred that compositions of the present invention comprise an amount of ligand which is effective at modulating the synthesis of bile acids. This amount, referred to herein as the “bile acid synthesis modulating amount” can be determined routinely for each identified ligand based upon its activity determined in vitro in human cells and in vivo in animal models. Bile acid modulating amounts can be confirmed in patients in need thereof by monitoring the effects of the ligand on cholesterol and/or lipid levels in the patient. Methods for monitoring cholesterol and lipid levels in a patient are well known and performed routinely by those skilled in the art.

The following non-limiting examples are provided to further illustrate the present invention.

### EXAMPLES

### Example 1

### Materials

Chenodeoxycholic acid, dexamethasone, and charcoal-stripped, delipidated calf serum were purchased from Sigma Chemical Co. (St. Louis, Mo.). DNA modifying enzymes, polymerases and restriction endonucleases were purchased from Roche Molecular Biochemicals (Indianapolis, Ind.). Charcoal, dextran-treated fetal bovine serum (FBS) was purchased from Hyclone Laboratories Inc. (Logan, Utah). The human hepatocellular carcinoma cell line HepG2 was obtained from the American Type Culture Collection (ATCC number HB-8065, Manassas, Va.). MATRIGEL was obtained from Becton Dickinson Labware (Bedford, Mass.). All other tissue culture reagents were obtained from Life Technologies Inc. (Gaithersburg, Md.).

### Example 2

### Animals

Male Fisher rats were obtained from Charles River Laboratories Inc. (Raleigh, N.C.) and maintained on a 12 hour light/12 hour dark cycle. Animals were allowed food and chow ad libitum. GW4064 (30 mg/kg) was administered by gavage twice a day for 7 days and the animals sacrificed by cervical dislocation 4 hours after final treatments. Livers were excised and snap-frozen in liquid nitrogen. Differential gene expression analysis was performed by Curagen Corp. (New Haven, Conn.).

### Example 3

### Plasmid Constructs

Expression plasmids for the human nuclear receptor-GAL4 chimeras were prepared by inserting amplified cDNAs encoding the ligand binding domains into a modified pSG5 expression vector (Stratagene, La Jolla, Calif.) containing the GAL4DBD (amino acids 1 to 147) and the Simian virus 40 (SV40) large T antigen nuclear localization signal (APKKKRKVG; SEQ ID NO: 1). The (UAS)₅-TK-CAT and (hsp27EcRE)₂-TK-LUC reporter constructs have been previously described (Lehmann et al. 1995. J. Biol. Chem. 270:12953-12956 and Forman, B. M. et al. 1995. Cell 81:687-693, respectively). p -actin-SPAP, an expression vector containing the human secreted placental alkaline phosphatase (SPAP) cDNA under the control of -actin promoter was used as an internal control in all transfections. The expression plasmids for human and mouse FXR (pSG5-hFXR and pSG5-mFXR, respectively) and human SRC-1 have been previously described (Kliewer, S. A. et al. 1998. Cell 92:73-82; Parks, D. J. et al. 1999. Science 284:1365-1368). The full-length coding regions for human LRH-1 (GenBank AB019246) and human SHP-1 (GenBank L76571) were amplified by PCR and cloned into pSG5, creating pSG5-hLRH-1 and pSG5-hSHP-1, respectively. A consensus Kozak sequence was created during amplification. The rat (bases −441 to +19) and human (−572 to +10) SHP-1 promoters were amplified by PCR and the fragments inserted into the BglII site of pGL3-Basic, a promoter-less luciferase reporter vector (Promega, Madison, Wis.). Site-directed mutagenesis of putative FXR/RXR binding sites in the rat and human SHP-1 promoters was performed using the Transformer mutagenesis system (Clontech, Palo Alto, Calif.) with the ratIP1 (bases −321 to −287, 5′-CCTGGTACAGCCTGGaaTAATAtaaCTGTTTATAC-3′; SEQ ID NO: 2) and humanIR1 (bases −304 to −270, 5′-CCTGGTACAGCCTGAaaTAATGtaTTGTTTATACC-3′; SEQ ID NO: 3) primers. Underlined residues are those which have been mutated from the wild-type sequence. Mutated constructs were verified to be free of non-specific base changes by sequencing. pGL3-rCYP7A (−1573/+36) contains bases −1573 to +36 of the rat CYP7A promoter (GenBank Z14108) inserted into the NheI site of pGL3-Basic. VP16-nuclear receptor chimeras contained the 80-amino acid herpes virus VP16 transactivation domain linked to the nuclear receptor ligand binding domain in a modified pSG5 expression vector.

### Example 4

### Transient Transfection Assays

Transient transfection of CV-1 cells was performed as described previously (Jones, S. A. et al. 2000. Mol. Endocrinol. 14:27-39). Typically, transfection mixes contained 2-5 ng receptor expression vector, 20 ng reporter construct, and 8 ng p -actin-SPAP. The amount of DNA used in each transfection was adjusted to 80 ng with carrier plasmid (pBluescript, Stratagene, La Jolla, Calif.). Cells were maintained for 24 hours in the presence of drug (added as a 1000× stock in dimethyl sulfoxide) in DMEM/F-12 nutrient mixture containing 10% charcoal-stripped, delipidated calf serum. An aliquot of medium was assayed for SPAP activity and the cells lysed prior to determination of luciferase expression. Luciferase activities were normalized to SPAP. HepG2 cells were maintained in DMEM/F-12 supplemented with 10% heat-inactivated FBS (Life Technologies, Inc., Gaithersburg, Md.). Plasmid DNA was transfected into HepG2 cells using FuGENE6 transfection reagent according to the manufacturer's instructions (Roche Molecular Biochemicals, Indianapolis, Ind.) Thus, 24 well culture plates (15 mm diameter) were inoculated with 7×10⁵ cells 24 hours prior to transfection. Cells were transfected overnight in serum-free DMEM/F-12 with 100 ng reporter construct, 32 ng p -actin-SPAP, and 0-400 ng receptor expression vectors (adjusted to 400 ng with carrier plasmid). Following transfection, the medium was aspirated and the cells cultured for a further 48 hours in DMEM/F-12 supplemented with 10% heat-inactivated FBS. SPAP and luciferase values were determined.

### Example 5

### Primary Culture of Human and Rat Hepatocytes and Northern Blot Analysis

Primary human hepatocytes and rat hepatocytes (1.5×10⁶ cells) were cultured on MATRIGEL-coated six well plates in serum-free Williams' E medium supplemented with 100 nM dexamethasone, 100 U/ml penicillin G, 100 μg/ml streptomycin, and insulin-transferrin-selenium (ITS-G, Life Technologies, Inc., Gaithersburg, Md.). Twenty-four hours after isolation, hepatocytes were treated with either GW4064 (0.1-10 μM) or chenodeoxycholic acid (1-100 μM) which were added to the culture medium as 1000× stocks in dimethyl sulfoxide. Control cultures received vehicle alone. Cells were cultured for a further 48 hours prior to harvest and total RNA isolated using a commercially available reagent (Trizol, Life Technologies Inc., Gaithersburg, Md.) according to the manufacturer's instructions. Total RNA (10 μg) was resolved on a 1% agarose/2.2 M formaldehyde denaturing gel and transferred to a nylon membrane (Hybond N+, Amersham Pharmacia Biotech Inc., Piscataway, N.J.). Blots were hybridized with ³²p-labeled cDNAs corresponding to human SHP-1, human CYP7A (bases 99 to 1564, GenBank M93133), mouse SHP-1 (bases 30 to 783, GenBank L76567), or rat CYP7A (bases 235 to 460, GenBank J05460). The SHP-1 cDNA used in these experiments encodes the full-length human SHP-1 protein (amino acids 1-260) as described in Seol et al. (1996 Science 272:1336), Subsequently, blots were stripped and reprobed with a radiolabeled -actin cDNA (Clontech, Palo Alto, Calif.).

### Example 6

### Electrophoretic Mobility-Shift Assay

Electrophoretic mobility shift assays (EMSA) were performed as previously described (Lehmann, J. M. et al. 1997. J. Biol. Chem. 272:3137-3140). HFXR and hRXR were synthesized from pSG5-hFXR and pSG5-hRXR expression vectors, respectively, using the TNTT7-coupled Reticulocyte System (Promega, Madison, Wis.). Unprogrammed lysate was prepared using the pSG5 expression vector (Stratagene, La Jolla, Calif.). Binding reactions contained 10 mM HEPES, pH 7.8, 60 mM KCl, 0.2% nonidet P-40, 6% glycerol, 2 mM dithiothreitol (DTT), 2 μg poly(dI-dC)*poly(dI-dC), and 1 μl each-of synthesized hFXR or hRXR . Control incubations received unprogrammed lysate alone. Reactions were pre-incubated on ice for 10 minutes prior to the addition of [³²p]-labeled double-stranded oligonucleotide probe (0.2 pmol). Competitor oligonucleotides were added to the pre-incubation at 5, 25 or 75-fold molar excess. Samples were held on ice for a further 20 minutes and the protein-DNA complexes resolved on a pre-electrophoresed 5% polyacrylamide gel in 0.5×TBE (45 mM Tris-borate, 1 mM EDTA) at room temperature. Gels were dried and autoradiographed at −70 C for 1 to 2 hours. The following double-stranded oligonucleotides were used as probes and competitors in EMSA: rSHP, 5′-gatcCCTGGGTTAATAACCCTGT-3′ (SEQ ID NO: 4); mSHP, 5′-gatcCCTGGGTTAATGACCCTGT-3′ (SEQ ID NO: 5); hSHP, 5′-gatcCCTGAGTTAATGACCTTGT-3′ (SEQ ID NO: 6); mI-BABP, 5′-gatcTTAAGGTGAATAACCTTGG-3′ (SEQ ID NO: 7); hI-BABP, 5′-gatcCCAGGTGAATAACCTCGG-3′ (SEQ ID NO: 8); mSHPmut, 5′-gatcCCTGGaaTAATGttCCTGT-3′ (SEQ ID NO: 9). Underlined residues are those which have been mutated from the wild-type sequence.

### Example 7

### GST Pull-Down Assays

GST-SHP-1 fusion protein was expressed in BL21(DE3)plysS cells and bacterial extracts prepared by one cycle of freeze-thaw of the cells in protein lysis buffer containing 50 mM Tris (pH 8.0), 250 mM KCl, 1% Triton X-100, 10 mM DTT and 1X Complete Protease Inhibitor (Roche Molecular Biochemicals, Indianapolis, Ind.) followed by centrifugation at 40,000×g for 30 minutes. Glycerol was added to the resultant supernatant to a final concentration of 10%. Lysates were stored at −80 C until use. [³⁵s]-labeled human LRH-1 or mouse pregnane X receptor (PXR), a negative control, were generated using TNT T7-coupled Reticulocyte System (Promega) in the presence of PRO-MIX (Amersham Pharmacia Biotech Inc., Piscataway, N.J.). Coprecipitation reactions included 25 μl lysate containing GST-SHP-1 fusion protein or control GST, 25 μl incubation buffer (50 mM KCl, 40 mM HEPES, pH 7.5, 5 mM —mercaptoethanol, 0.1% TWEEN 20, and 1% non-fat dry milk), and 5 μl [³⁵S]-labeled LRH-SHP-1 or PXR. The mixtures were incubated for 25 minutes with gentle rocking at 4 C prior to the addition of 20 μl glutathione-sepharose 4B beads (Amersham Pharmacia Biotech Inc., Piscataway, N.J.) that had extensively washed in protein lysis buffer. Reactions were incubated at 4 C with gentle rocking for an additional 20 minutes. The beads were pelleted at 3000 rpm in a microfuge and washed 4 times with protein incubation buffer. Following the final wash, the beads were resuspended in 25 μl of 2× SDS-PAGE sample buffer containing 50 mM DTT. Samples were heated to 100 C for 5 minutes and loaded onto 10% Bis-Tris PAGE gel. Autoradiography was performed overnight.

All of the references cited in this application are herein incorporated by reference.

Other objects, features, advantages and aspects of the present invention will become apparent to those of skill in the art from the above description and the following claims. It should be understood, therefore, that the above description including the specific examples as well as the following claims, while indicating preferred embodiments of the invention, are given by way of illustration only. Various changes and modifications within the spirit and scope of the disclosed invention which will become readily apparent to those skilled in the art from reading this disclosure are therefore also encompassed by this application.

What is claimed is:

1. A method for identifying compounds that modulate bile acid synthesis comprising assessing the ability of a compound to act as a ligand for short heterodimerizing partner-1 or liver receptor homologue-1, the ability of the compound to act as a ligand for one of these receptors being indicative of the compound being a modulator of bile acid synthesis.

2. The method of claim 1 wherein the ability of the ligand to modulate the interaction of short heterodimerizing partner-1 with liver receptor homologue-1 is assessed.

3. A method for modulating bile acid synthesis in a patient in need thereof comprising administering to a patient a composition comprising a ligand for short heterodimerizing partner-1 or liver receptor homologue-1.

4. The method of claim 3 wherein the composition comprises a ligand which modulates the interaction of short heterodimerizing partner-1 with liver receptor homologue-1.

5. The method of claim 3 wherein the composition comprises a bile acid synthesis modulating amount of ligand.

6. The method of claim 3 wherein cholesterol or lipid homeostasis is modulated.

7. A composition for modulating bile acid synthesis comprising a ligand for short heterodimerizing protein-1 or liver receptor homologue-1.

8. The composition of claim 7 wherein the ligand modulates the interaction of short heterodimerizing protein-1 with liver receptor homoloque-1.

9. The composition of claim 7 comprising a bile acid synthesis modulating amount of ligand.
